(19) United States
(12) Patent Application Publication
(10) Pub. No.: US 2023/0233598 A1
(43) Pub. Date: Jul. 27, 2023

(54) Marine Bacterial Exopolysaccharide Derivatives and Uses Thereof in the Treatment of Mucopolysaccharidoses

(71) Applicants: Institut Francais de Recherche Pour l'Exploitation de la Mer (IFREMER), Issy les Moulineaux (FR); Fondation Sanfilippo Suisse, Geneva (CH)

(72) Inventors: Sylvia Colliec-Jouault, Nantes (FR); Corinne Sinquin, Nantes (FR); Agata Zykwinska, Saint Herblain (FR); Ariane De Agostini, Genthod (CH); Jean-Christophe Tille, Geneve (CH); Noémi Veraldi, Geneve (CH); Isabelle Dentant-Quadri, Annemasse (FR); Jennifer Mbosso Beffort, Gland (CH); Nawel Zouggari, Geneva (CH)

(73) Assignees: Institut Francais de Recherche Pour l'Exploitation de la Mer (IFREMER), Issy les Moulineaux (FR); Fondation Sanfilippo Suisse, Geneva (CH)

(21) Appl. No.: 18/013,346

(22) PCT Filed: Jul. 1, 2021
(86) PCT No.: PCT/EP2021/068202

§ 371 Date: Dec. 28, 2022

(30) Foreign Application Priority Data
Jul. 2, 2020 (EP) 20183661.6

(51) Int. Cl. A61K 31/737; A61K 35/74; A61K 45/06; A61P 3/00

(52) U.S. Cl. CPC A61K 31/737; A61K 35/74; A61K 45/06; A61P 3/00

(57) ABSTRACT

The invention provides low-molecular-weight over-sulfated polysaccharides with anti-heparanase activity prepared from marine native exopolysaccharides excreted by mesophilic marine bacteria from a deep-sea hydrothermal environment and relates to the use of these low-molecular-weight over-sulfated polysaccharides for the prevention or treatment of mucopolysaccharidoses, in particular mucopolysaccharidoses type III (Sanfilippo syndrome).

### RELATED APPLICATION

The present application claims priority to European Patent Application No. EP 20 183 661.6 filed on Jul. 2, 2020, which is incorporated herein by reference in its entirety.

### BACKGROUND OF THE INVENTION

The mucopolysaccharidoses (MPSs) are a group of rare inherited metabolic disorders within the larger lysosomal storage disease (LSD) family. Overall incidence is 1 in 25,000 to 30,000 live births. However, because mucopolysaccharidoses, especially the milder forms of the diseases, often go unrecognized, these disorders are underdiagnosed or misdiagnosed, making it difficult to determine their true frequency in the general population. MPSs are characterized by a deficiency (absence or malfunctioning) of any one of eleven specific lysosomal enzymes involved in the metabolism (catabolism or degradation) of glycosaminoglycans (GAGs)—long unbranched polysaccharides that play an essential role in connective tissue biology and cellular crosstalk. Such lysosomal enzyme deficiency leads to an accumulation of both non-degraded and partially degraded GAGs within the lysosomes resulting in permanent, progressive cellular damage that gives rise to a multisystemic disease. Individuals with MPS disorders share many similar symptoms such as multiple organ involvement, distinctive “coarse” facial features, and abnormalities of the skeleton, especially joint problems. Additional findings include short stature, heart abnormalities, breathing irregularities, liver and spleen enlargement and/or neurological abnormalities. The severity of the different MPS disorders varies greatly among affected individuals, even among those with the same type of MPS and even among individuals of the same family. Although each MPS differs clinically, most patients generally experience a period of normal development followed by a decline in physical and/or mental function. The MPSs include the following different types: MPS I-H/S (Hurler/Scheie syndrome), MPS I-H (Hurler syndrome), MPS I-S(Scheie syndrome), MPS II (Hunter syndrome), MPS III (Sanfilippo syndrome), MPS IV (Morquio syndrome), MPS IX (hyaluronidase deficiency or Natowicz syndrome), MPS VII (Sly syndrome) and MPS VI (Maroteaux-Lamy syndrome). Currently, no specific treatment is available for supportive care and treatment of complications and treatment strategy is needed for a large subgroup of MPS patients.

Of the MPS disorders, mucopolysaccharidosis type III, also known as Sanfilippo syndrome, is the most common. MPS III is composed of four different subtypes: A, B, C and D, each subtype being caused by the deficiency of one of four enzymes involved in the degradation of heparan sulfate. Overall incidence of MPS III varies between 0.28 and 4.1 per 100,000 live births. The incidence of the different subtypes has a very uneven geographic distribution; however, types A and B are always more common than types C and D. In all MPS III subtypes, central nervous system (CNS) involvement predominates (neurodegeneration, progressing dementia, hyperactivity, seizures and behavioral disturbances), but other symptoms can also be present such as skeletal pathology that affect growth and causes degenerative joint disease, hepatosplenomegaly, macrocrania, and hearing loss. Except in attenuated patients, death usually occurs in the second decade, children with MPS III subtype A having the shorter survival rate. There is currently no cure or standard treatment for patients with Sanfilippo syndrome. In the absence of effective therapies, patient care is limited to symptom management and palliative support. MPS III disorders are sufficiently debilitating to patients and challenging to parents and caregivers to warrant attention and research. Gene therapy, bone marrow transplant, chaperon molecules, substrate deprivation therapy and intrathecal enzyme therapy are among the most active therapeutic research areas.

Although several developments raise hope that therapeutic interventions, halting the devastating mental and behavioral deterioration, might be feasible at some point in the future, there is currently no effective therapy available for MPS III and the other MPSs. Thus, there is still a need in the art for therapeutic options in the treatment of mucopolysaccharidosis disorders.

### SUMMARY OF THE INVENTION

The present Inventors have shown that low molecular weight over-sulfated polysaccharides obtained from a marine native exopolysaccharide (EPS) excreted by the strain GY785 (an Alteromonas infernus species of the Alteromonas genus) or from the strain HE800 (a Vibrio diabolicus species of the Vibrio genus) exhibit anti-heparanase (HPSE) activity. The low molecular weight over-sulfated polysaccharides proved to be able to affect heparan sulfate (HS) turnover thus leading to an incomplete degradation of intracellular HS in fibroblasts derived from a mouse model of mucopolysaccharidosis of type IIIA. Heparanase is the first enzyme to degrade heparan sulfate in its catabolic pathway, cleaving large segments of the HS chain that are subsequently depolymerized in lysosomes by exoglycosidases. Although the mechanism is not fully understood, it is believed that intact HS chains, that are not cleaved by HPSE (due to the inhibitory effects of the low molecular weight over-sulfated polysaccharides), cannot enter the lysosomal degradative pathway and are redirected to the extracellular space with clearance in blood and urine. Thus, cells can be preserved from toxic lysosomal HS accumulation. Treatment with the low molecular weight over-sulfated polysaccharides could alleviate the symptoms due to lysosomal overload with incompletely degraded heparan sulfate.

Accordingly, in a first aspect, the present invention relates to a low molecular weight over-sulfated polysaccharide with anti-heparanase activity for use in the prevention or treatment of a mucopolysaccharidosis in a subject, wherein said low molecular weight over-sulfated polysaccharide with anti-heparanase activity is a derivative of a native exopolysaccharide (EPS) excreted by a mesophilic marine bacterium from a deep-sea hydrothermal environment and wherein said low molecular weight over-sulfated polysaccharide is obtained using a method comprising the following steps:

(a) a step consisting of free-radical depolymerization of the marine native EPS from the strain GY785 of the Alteromonas genus or from the strain HE800 of the Vibrio diabolicus genus so as to obtain a depolymerized EPS having a molecular weight of 5,000 to 100,000 g/mol;

(b) a subsequent step consisting of sulfation of the depolymerized EPS to obtain an over-sulfated depolymerized EPS, comprising adding to the depolymerized EPS at least one sulfation agent in an amount sufficient to obtain a sulfated polysaccharide having a degree of sulfate-group substitution of between 10% and 55% by weight relative to the total weight of the over-sulfated depolymerized EPS; and

(c) a subsequent step consisting of isolating the low molecular weight over-sulfated polysaccharide from the over-sulfated depolymerized EPS, wherein the low-molecular-weight over-sulfated polysaccharide has a molecular weight of between about 5,000 and about 16,000 g/mol.

In certain embodiments, in step (a) of the preparation method defined above, the free-radical depolymerization is performed on the native GY785 EPS excreted by the strain GY785 and said low molecular weight over-sulfated polysaccharide with anti-heparanase activity has a molecular weight of between about 6 kDa and about 10 kDa or between about 7 kDa and about 9 kDa, and a degree of sulfate-group substitution of between about 30% and about 40% by weight relative to the total weight of the over-sulfated polysaccharide.

For example, said low molecular weight over-sulfated polysaccharide with anti-heparinase activity may be GYS8, which has a molecular weight of about 8 kDa, and a degree of sulfate-group substitution of about 36% by weight relative to the total weight of the over-sulfated polysaccharide.

In certain embodiments, in step (a) of the preparation method defined above, the free-radical depolymerization is performed on the native HE800 EPS excreted by the strain HE800 and wherein said low molecular weight over-sulfated polysaccharide with anti-heparanase activity has a molecular weight of between about 3 kDa and about 7 kDa or between about 4 kDa and about 6 kDa, and a degree of sulfate-group substitution of between about 45% and about 55% by weight relative to the total weight of the over-sulfated polysaccharide.

For example, said low molecular weight over-sulfated polysaccharide with anti-heparanase activity may be HE5.1, which has a molecular weight of about 5.1 kDa, and a degree of sulfate-group substitution of about 50% by weight relative to the total weight of the over-sulfated polysaccharide.

In certain embodiments, the step of isolating the low molecular weight over-sulfated polysaccharide from the over-sulfated depolymerized EPS is carried out by fractionation, in particular fractionation performed by size exclusion chromatography.

In certain embodiments, the mucopolysaccharidosis is a mucopolysaccharidosis type III. The mucopolysaccharide type III may be of subtype A, subtype B, subtype C or subtype D.

In another aspect, the present invention provides a pharmaceutical composition comprising a therapeutically effective amount of the low molecular weight over-sulfated polysaccharide with anti-heparanase activity as defined herein and at least one pharmaceutically acceptable carrier or excipient for use in the prevention or treatment of a mucopolysaccharidosis in a subject.

In certain embodiments, the mucopolysaccharidosis is a mucopolysaccharidosis type III. The mucopolysaccharide type III may be of subtype A, subtype B, subtype C or subtype D.

These and other objects, advantages and features of the present invention will become apparent to those of ordinary skill in the art having read the following detailed description of the preferred embodiments.

### BRIEF DESCRIPTION OF THE DRAWING

FIG. 1: Schematic Model of Heparan Sulfate Proteoglycans and Heparanase Trafficking.

FIG. 2: Effect of Treatment on Total Proteoglycans. The graph reports the total amount of proteoglycans in control and treated cells (fibroblasts derived from a mouse model of MPSIIIA.) For each exopolysaccharide derivative (HES5.1 (A5_3) and GYS8 (A5_4)), the average of two independent experiments is reported ±S.D.

FIG. 3: Distribution of Proteoglycans in Control and Treated Cells. The graph reports the radioactivity measured in extracellular and intracellular compartments following the first purification step, corresponding to total proteoglycans. For each exopolysaccharide derivative, the average of two independent experiments is reported ±S.D.

FIG. 4: Distribution of Heparan Sulfate in Control and Treated Cells. The graph reports the percentage of heparan sulfate (HS) in the corresponding proteoglycans (PGs) in extracellular and intracellular compartments. The PGSs in each compartment are considered as 100%. For each compound ((A) A5_4 (GYS8) and (B) A5_3 (HES5.1)), the average of two independent experiments is reported ±S.D.

FIG. 5: PAGE-NaCl Profile of Intracellular and Extracellular Heparan Sulfate (HS) in MPSIIIA cells. HS was isolated from control cells and analyzed by PAGE. The standards are detected with Azure A 0.08% while HS from MPSIIIA cells was detected by autoradiography. On the left are reported the profiles of intracellular and extracellular HS obtained using the software ImageJ and the corresponding gel is shown on the right.

FIG. 6: Profiles Obtained by GFC. (A) The first graph presents the standard curve. The molecular weights of the standards are reported. (B) The second graph presents the profiles of intracellular and extracellular HS in control MPSIIIA cells.

FIG. 7: PAGE-NaCl Profile of Intracellular and Extracellular HS in MPSIIIA Cells Treated with A5_3 (HES5.1). HS was isolated from control and treated cells, analyzed by PAGE NaCl and detected by autoradiography.

FIG. 8: PAGE-NaCl Profile of Intracellular and Extracellular HS in MPSIIIA Cells Treated with A5_4 (GYS8). HS was isolated from control and treated cells, analyzed by PAGE NaCl and detected by autoradiography.

FIG. 9: GFC Profiles of HS from Control (Untreated) Cells and Treated with 20 μg/ml A5_3 (HES5.1). (A) Superimposition of the profiles of extracellular HS from control and treated cells. (B) Superimposition of the profiles of intracellular HS from control and treated cells. Treatment causes a shift towards the molecular weight (MW) corresponding to that of extracellular HS.

FIG. 10: GFC Profiles of HS from Control (Untreated) Cells and Treated with 20 μg/ml A5_4 (GYS8). (A) Superimposition of the profiles of extracellular HS from control and treated cells. (B) Superimposition of the profiles of intracellular HS from control and treated cells. Treatment causes a shift towards the molecular weight (MW) corresponding to that of extracellular HS.

FIG. 11: PAGE Profiles of HS from MPSIIIA Cells Treated with A5_3 (HES5.1). (A) Intracellular HS from cells treated with 0, 20, 50, and 100 μg/ml A5_3. (B) Extracellular HS from cells treated with 0, 20, 50, and 100 μg/ml A5_3.

### DEFINITIONS

As used herein, the term “subject” refers to a human or another mammal (e.g., primate, dog, cat, goat, horse, pig, mouse, rat, rabbit, and the like), that can develop a mucopolysaccharidosis, but may or may not be suffering from the disease. Non-human subjects may be transgenic or otherwise modified animals. In many embodiments of the present invention, the subject is a human being. In such embodiments, the subject is often referred to as an “individual” or a “patient”. These terms do not denote a particular age, and thus encompass new-borns, children, teenagers, and adults. The term “patient” more specifically refers to an individual suffering from a disease. Thus, the term “mucopolysaccharidosis patient” refers to an individual suffering from (i.e., diagnosed with) a mucopolysaccharidosis.

As used herein, the term “inhibit” means to prevent something from happening, to delay occurrence of something happening, and/or to reduce the extent or likelihood of something happening. Thus, the terms “heparanase inhibitor” and “HPSE inhibitor”, which are used herein interchangeably, are intended to refer to a molecule, compound or agent that inhibits (i.e., prevents, reduces, and/or delays) the normal functioning of heparanase. The term “with anti-heparanase activity”, when used herein to characterize a molecule, compound or agent, refers to a molecule, compound or agent that is a heparinase inhibitor.

The term “treatment” is used herein to characterize a method or process that is aimed at (1) delaying or preventing the onset of a disease or condition (here a mucopolysaccharidosis); (2) slowing down or stopping the progression, aggravation, or deterioration of the symptoms of the disease or condition; (3) bringing about amelioration of the symptoms of the disease or condition; or (4) curing the disease or condition. A treatment may be administered after initiation of the disease or condition, for a therapeutic action. Alternatively, a treatment may be administered prior to the onset of the disease or condition, for a prophylactic or preventive action. In this case, the term “prevention” is used.

A “pharmaceutical composition” is defined herein as comprising an effective amount of a low molecular weight (LMW) over-sulfated polysaccharide derivative with anti-heparanase activity according to the invention, and at least one pharmaceutically acceptable carrier or excipient.

As used herein, the term “therapeutically effective amount” refers to any amount of a molecule, compound, agent, or composition that is sufficient to fulfil its intended purpose(s), e.g., a desired biological or medicinal response in a cell, tissue, system or subject.

The term “pharmaceutically acceptable carrier or excipient” refers to a carrier medium which does not interfere with the effectiveness of the biological activity of the active ingredient(s) and which is not excessively toxic to the host at the concentration at which it is administered. The term includes solvents, dispersion media, coatings, antibacterial and antifungal agents, isotonic agents, and adsorption delaying agents, and the like. The use of such media and agents for pharmaceutically active substances is well known in the art (see for example “Remington's Pharmaceutical Sciences”, E. W. Martin, 18^{th} Ed., 1990, Mack Publishing Co.: Easton, Pa., which is incorporated herein by reference in its entirety).

The terms “approximately” and “about”, as used herein in reference to a number, generally include numbers that fall within a range of 10% in either direction of the number (greater than or less than the number) unless otherwise stated or otherwise evident from the context (except where such number would exceed 100% of a possible value).

### DETAILED DESCRIPTION OF CERTAIN PREFERRED EMBODIMENTS

As mentioned above, the present invention provides low molecular weight over-sulfated polysaccharides with anti-heparanase activity that are derivatives of native exopolysaccharides excreted by a mesophilic marine bacterium from a deep-see hydrothermal environment and relates to the use of these low molecular weight over-sulfated polysaccharides in the prevention or treatment of a mucopolysaccharidosis in a subject, in particular in the prevention or treatment of a mucopolysaccharidosis of type III.

### I—Low Molecular Weight Over-Sulfated Exopolysaccharide Derivatives

The low molecular weight over-sulfated polysaccharides used in the present invention are derivatives of two native exopolysaccharides (EPSs), HE800 EPS and GY785 EPS, that are excreted by mesophilic marine bacteria from a deep-sea hydrothermal environment. In recent years, there has been a growing interest in the isolation and identification of new polysaccharides of marine origin that might have new applications in diverse industries. They compete with polysaccharides from other sources such as seaweeds, crustaceans, animals or plants. Interest in mass culture of microorganisms from the marine environment has increased considerably, representing an innovative approach to the biotechnological use of under-exploited resources. Marine bacterial EPSs and derivatives thereof have some great advantages as therapeutic compounds because they can be produced at viable economic cost, in controlled conditions in agreement with Good Manufacturing Practices and exhibit a very low risk for patients to be infected by a non-conventional transmissible agent, such as prions or emerging viruses, due to a large “species-barrier”.

Marine bacteria from deep-sea hydrothermal vent environments, belonging to three main genera (Vibrio, Alteromonas and Pseudoalteromonas), have demonstrated their ability to produce unusual extracellular polymers in an aerobic carbohydrate-supplemented medium. The excreted exopolysaccharides present original structural features that can be modified to design bioactive compounds and improve their specificity (Rehm et al., Rev. Microbiol., 2010, 8: 578-592; Colliec-Jouault et al., Handbook of Exp. Pharmacol., 2012, 423-449; Delbarre-Ladrat et al., Microorganisms, 2017, 5(3): 53). In particular, the first EPS-producing species of Vibrio to be isolated from an active deep-sea hydrothermal vent sample was named Vibrio diabolicus (Raguenes et al., Int. J. Syst. Bacteriol., 1997, 47: 989-995). It produces a high molecular weight (>10⁶ g/mol—Rougeaux et al., J. Carbohydr. Res., 1999, 322: 40-45) exopolysaccharide, called HE800 EPS, which consists of a linear tetrasaccharide repeating unit: two glucuronic acid residues, one N-acetylated glucosamine residue and one N-acetylated galactosamine residue. Another bacterium, named Alteromonas infernus—a new species of the genus Alteromonas—was also isolated in deep-sea sediments of the Guaymas basin (Gulf of California) (Raguenes et al., J. Appl. Microbiol., 1997, 82: 422-430). This bacterium Alteromonas infernus produces a water-soluble EPS, called GY785 EPS, a branched heteropolysaccharide with a nonasaccharide repeating unit comprising: four glucose residues, two galactose residues, two glucuronic acid residues and one galacturonic acid residue bearing a sulfate group at the C2 position (Roger et al., Carbohydr. Res., 2004, 339: 2371-2380; Guezennec et al., Carbohydr. Polym., 1998, 37: 19-24).

Low molecular weight (LMW) over-sulfated polysaccharide derivatives from the marine native exopolysaccharides, HE800 EPS and GY785 EPS, have previously been prepared by the present Inventors (Colliec-Jouault et al., Biochim. Biophys. Acta, 2001, 1528: 141-151; WO 2006/003290; WO 2007/066009; WO 02/02051; Guezennec J. et al. in Carbohydrate Polymers 1998, 37(1): 19-24; Senni et al., Mar. Drugs, 2013, 11: 1351-1369; Merceron et al., Stem Cells, 2012, 30: 471-480; Senni et al., Mar. Drugs, 2011, 9: 1664-1681; Heymann et al., Molecules, 2016, 21: 309) using a first step of radical depolymerization followed by a sulfation reaction, thereby generating bioactive molecules having a molecular weight <30 kg/mol (30,000 Da). In the practice of the present invention, a low molecular weight over-sulfated polysaccharide is prepared using a similar method, said method comprising:
(a) a step consisting of free-radical depolymerization of a marine native exopolysaccharide (EPS) from the strain GY785 of the Alteromonas genus or from the strain HE800 of the Vibrio genus so as to obtain a depolymerized EPS having a molecular weight of 5,000 to 100,000 g/mol;
(b) a subsequent step consisting of sulfation of the depolymerized EPS to obtain an over-sulfated depolymerized EPS, comprising adding to the depolymerized EPS at least one sulfation agent in an amount sufficient to obtain a sulfated polysaccharide having a degree of sulfate-group substitution of between about 10% and about 55% by weight relative to the total weight of the over-sulfated depolymerized EPS; and
(c) a subsequent step consisting of isolating the low molecular weight over-sulfated polysaccharide from the over-sulfated depolymerized EPS, wherein the low molecular weight over-sulfated polysaccharide has a molecular weight of between about 5,000 and about 16,000 g/mol.

In certain embodiments, the depolymerized EPSs obtained after step (a) are lyophilized.

In other embodiments, step (b) of the process is followed by a dialysis step.

During the first depolymerization step, the native EPS can be used in a liquid form, i.e. as it is excreted by the bacteria into the culture medium. Preferably, the culture medium is centrifuged and only the supernatant that contains the native EPS and that is free of bacterial debris is collected. The native EPS can be collected by any suitable technique known to those skilled in the art, such as for example membrane ultrafiltration, and can then optionally be lyophilized as is or in the form of an addition salt.

The step consisting of free-radical depolymerization of the native EPS is preferably carried out by addition of a solution of an oxidizing agent to a reaction mixture comprising the native EPS, preferably in the presence of a metal catalyst. The oxidizing agent is preferably chosen from peroxides, in particular hydrogen peroxide, and peracids, especially peracetic acid and 3-chloroperbenzoic acid. The addition is preferably carried out continuously and with stirring for a period of between 30 minutes and 10 hours. The reaction mixture is preferably maintained at a pH of between 6 and 8, for example by addition of a basifying agent such as sodium hydroxide, and at a temperature of between approximately 30° C. and 70° C. throughout the duration of the free-radical depolymerization reaction.

According to a specific embodiment of the present invention, in this step, the native EPS is present in the reaction mixture at a concentration of between about 2 mg/ml and about 10 mg/ml of reaction mixture.

In preferred embodiments, the oxidizing agent is a solution of hydrogen peroxide (H₂O₂) preferably having a concentration of between about 0.1% and about 0.5% by weight, preferably of the order of 0.1% to 0.2% by weight, and is added at a flow rate of V1/1000 to V1/10 ml/minute, preferably V1/50 and V1/500 ml/minute, and more preferably of the order of V1/100 ml/minute, wherein V1 is the volume of the reaction medium containing a marine exopolysaccharide (EPS) to which a solution of hydrogen peroxide is added.

The metal catalysts that can be used during the depolymerization step are preferably chosen from Cu²⁺, Fe²⁺ and Cr³⁺ ions and the Cr₂O₇²⁻ anion, as described in particular in European patent application EP 0 221 977. According to a specific embodiment, the metal catalyst is present in the reaction mixture at a concentration of between about 10⁻³ M and about 10⁻¹ M, and preferably at a concentration of between about 0.001 M and about 0.05 M.

The free-radical depolymerization process according to the invention and as described above makes it possible to obtain, in a single step and with a good yield, homogeneous, low molecular weight polysaccharide derivatives. In the context of the present invention, the term “homogeneous derivatives” is intended to mean derivatives which, when assessed using high performance size exclusion chromatography, exhibit a single main peak representing a predominant population of polysaccharide chains that are homogeneous with respect to size, characterized by a polydispersity index I (Mw/Mn)<5, where Mw is the weight-average molecular weight and Mn is the number-average molecular weight.

In certain embodiments, when the depolymerization reaction is over, the polysaccharide derivatives obtained are reduced using a reducing agent, so as to stabilize the chains, the reducing ends of which are very reactive, and in particular to avoid chain hydrolysis by the “peeling” reaction. The nature of the reducing agents that can be used to this effect is not essential. In particular, the reducing agent may be sodium borohydride.

The metal catalyst used in the depolymerization step can be eliminated at the end of the depolymerization reaction, (or at the end of the reduction reaction if a reduction step is carried out) using any suitable method, for example by ion exchange chromatography, preferably a weak cation exchange resin passivated beforehand, or by treatment with EDTA (ethylenediaminetetraacetic acid).

The polysaccharide derivatives resulting from the depolymerization and/or from the reduction can, if necessary, be recovered by any suitable technique well known to those skilled in the art, such as, for example, by membrane ultrafiltration or dialysis. Then, they are lyophilized and fractionated by size exclusion chromatography to increase their purity required to improve the subsequent sulfation step. Finally, the purified polysaccharide derivatives are conditioned in salt form by addition of a weak or strong base that may be chosen, for example, from pyridine, triethylamine, tributylamine, tetrabutylammonium hydroxide and sodium hydroxide. This lyophilized salt may be prepared, for example, by elution of an aqueous solution of the polysaccharide derivatives at a concentration of between 1 and 8 mg/ml on an ion exchange resin column such as, for example, those sold under the name DOWEX® by the company Dow Chemical. The eluate is collected as long as the pH remains acid, for example less than 5, then the pH is subsequently adjusted to approximately 6.5 with the desired base as defined above. The polysaccharide derivatives in the form of a salt are then ultra-filtered and lyophilized.

The lyophilized polysaccharide derivatives, possibly in the form of an addition salt, are preferably dissolved in an anhydrous solvent at the beginning of the sulfation step. The solvent is preferably chosen from dimethylformamide (DMF), dimethyl sulfoxide (DMSO), formamide, and mixtures thereof. The amount of polysaccharide derivatives present in the anhydrous solvent may be between approximately 1 mg/ml and 10 mg/ml, preferably between about 1 mg/ml and about 5 mg/ml, and even more preferably this amount is about 2.5 mg/ml. The dissolution of the EPS in the anhydrous solvent is preferably carried out, with stirring, at room temperature for about 1 hour to about 2 hours and then at a temperature of between 40° C. and 50° C., preferably at a temperature of about 45° C. for about 2 hours under argon or azote with molecular sieves.

The one or more chemical sulfation agents used during the sulfation step can be added to the depolymerized and/or reduced EPSs that are in lyophilized form or in the form of a solution.

The sulfation agents are preferably chosen from complexes of pyridine sulfate (free or coupled to a polymer), of dimethylformamide sulfate, triethylamine sulfate and of trimethylamine sulfate. The one or more chemical sulfation agents are added to the solution of polysaccharide derivatives in a weight amount preferably representing from about 4 to about 6 times, and even more preferably about 5 times, the mass of polysaccharide derivatives in solution. The chemical sulfation reaction is then preferably carried out with stirring for a period of between 2 and 24 hours depending on the desired degree of sulfation. When the desired degree of sulfation is reached, the sulfation reaction is stopped after cooling of the reaction medium:
either by precipitation in the presence of sodium-chloride-saturated acetone or of methanol, and then dissolution of the precipitate in water;
or, preferably, by addition of water in a proportion preferably equal to 1/10 of the reaction volume and adjustment of the pH of the reaction medium to 9 with a basifying agent such as, for example, sodium hydroxide (3 M).

According to certain embodiments, the solution of sulfated polysaccharide derivatives is preferably dialyzed in order to remove the various salts, and then lyophilized. The final product (i.e., the low molecular weight, over-sulfated polysaccharide), typically with an accurate molecular weight and a low polydispersity index, is obtained by isolation from the low molecular weight depolymerized EPS obtained. Isolation may be performed by any suitable method known in the art. Preferably, isolation is carried out by fractionation performed by size exclusion chromatography.

The low molecular weight over-sulfated polysaccharides according to the present invention have a low polydispersity index of less than 5, preferably of 1.5 to 4, more preferably of less than 2. The polydispersity index (PDI), as used herein, is a measure of the distribution of molecular mass of the EPS derivatives. The PDI calculated is the weight average molecular weight divided by the number average molecular weight. PDI is typically measured by size-exclusion chromatography.

A low molecular weight over-sulfated polysaccharide according to the invention has a degree of sulfate-group substitution of between 10% and 55% by weight relative to the total weight of the sulfated polysaccharide derivative. In certain embodiments, the degree of sulfate-group substitution is of between 10% and 40%, of between 20% and 45% or of between 20% and 40%. In other embodiments, the degree of sulfate-group substitution is of between 30% and 60%, of between 40% and 55% or around 50%.

In certain embodiments, the low molecular weight over-sulfated polysaccharide is prepared from the native GY785 EPS, excreted by the strain GY785 (a Alteromonas infernus species of the Alteromonas genus), has a molecular weight of between about 6 kDa and about 10 kDa or between about 7 kDa and about 9 kDa, and a degree of sulfate-group substitution of between about 30% and about 40% by weight relative to the total weight of the over-sulfated polysaccharide. In a particular embodiment, the low molecular weight over-sulfated polysaccharide is GYS8, which is prepared from the native GY785 EPS, has a molecular weight of about 8 kDa, and a degree of sulfate-group substitution of about 36% by weight relative to the total weight of the over-sulfated polysaccharide.

In other embodiments, the low molecular weight over-sulfated polysaccharide is prepared from the native HE800 EPS excreted by the strain HE800 (a Vibrio diabolicus species of the Vibrio genus), has a molecular weight of between about 3 kDa and about 7 kDa or between about 4 kDa and about 6 kDa, and a degree of sulfate-group substitution of between about 45% and 55% by weight relative to the total weight of the over-sulfated polysaccharide. In a particular embodiment, the low molecular weight over-sulfated polysaccharide is HES5.1, which is prepared from the native HE800 EPS, has a molecular weight of about 5.1 kDa, and a degree of sulfate-group substitution of about 50% by weight relative to the total weight of the over-sulfated polysaccharide.

### II—Uses of the Low Molecular Weight Over-Sulfated Polysaccharides

### 1—Indications

The present Inventors have demonstrated that the low molecular weight over-sulfated polysaccharides described herein are heparanase (HSPE) inhibitors (i.e., exhibit anti-heparanase activity). Thus, due to their anti-HSPE activity, the low molecular weight over-sulfated polysaccharides, as defined above, in particular HES5.1 and GYS8, may be used in the treatment or prevention of a mucopolysaccharidosis in a subject, preferably a mucopolysaccharidosis of type III. The terms “mucopolysaccharidosis” and “MPS” are used herein interchangeably. They refer to a subgroup of lysosomal storage disorders characterized by the accumulation and storage of glycosaminoglycans (GAGs) within lysosomes. The mucopolysaccharidosis may be MPS I-H/S (Hurler/Scheie syndrome), MPS I-H (Hurler syndrome), MPS I-S(Scheie syndrome), MPS II (Hunter syndrome), MPS III (Sanfilippo syndrome), MPS IV (Morquio syndrome), MPS IX (hyaluronidase deficiency or Natowicz syndrome), MPS VII (Sly syndrome) or MPS VI (Maroteaux-Lamy syndrome). Preferably, the mucopolysaccharidosis is MPS III (Sanfilippo syndrome). MPS III is characterized by the presence of undegraded heparan sulfate due to the deficit of one of the four enzymes necessary for its catabolism, responsible for one of the four subtypes of MPS III: type IIIA (heparan sulfamidase), type IIIB (alpha-N-acetylglucosaminidase), type IIIC (alpha-glucosaminide-N-acetyltransferase) and type IIID (N-acetylglucosamine-6-sulfatase).

Methods of treatment of the present invention may be accomplished using a low molecular weight over-sulfated polysaccharide as described herein or a pharmaceutical composition thereof. These methods generally comprise administration of an effective amount of the low molecular weight over-sulfated polysaccharide (as defined above, in particular HES5.1 or GYS8), or a pharmaceutical composition thereof, to a subject in need thereof. Administration may be performed using any of the methods known to one skilled in the art. In particular, the low molecular weight over-sulfated polysaccharide, or a composition thereof, may be administered by any of various routes including, but not limited to, aerosol, parenteral, oral or topical route.

Preferably, the subject is a MPS III patient. In the practice of the present invention, the MPS III disorder may be of subtype A, B, C or D. In certain embodiments, the MPS III disorder is MPS IIIA or MPS IIIB.

In general, the low molecular weight over-sulfated polysaccharide, or a composition thereof, will be administered in an effective amount, i.e., an amount that is sufficient to fulfill its intended purpose. The exact amount of the low molecular weight over-sulfated polysaccharide, or pharmaceutical composition, to be administered will vary from subject to subject, depending on the subtype of the MPS III disorder, the age, sex, weight and general health condition of the subject to be treated, the desired biological or medical response and the like. In certain embodiments, an effective amount is one that prevents, delays and/or reduces the likelihood of at least one symptom associated with the MPS disorder. For example, in the case of a MPS III disorder, the symptom may be behavioral problems (temper tantrums, hyperactivity, destructiveness, aggressive behavior, pica, sleep disturbance, seizures), walking problems, stiff joints, vision and/or hearing loss, communication difficulties, etc. The effects of a treatment according to the invention may be monitored using any of the diagnostic assays, tests and procedures known in the art.

In certain embodiments, a low molecular weight over-sulfated polysaccharide described herein, or a composition thereof, is administered alone according to a method according to the present invention. In other embodiments, the low molecular weight over-sulfated polysaccharide, or a composition thereof, is administered in combination with at least one additional therapeutic agent or therapeutic procedure. The low molecular weight over-sulfated polysaccharide, or a composition thereof, may be administered prior to administration of the therapeutic agent or therapeutic procedure, concurrently with the therapeutic agent or procedure, and/or following administration of the therapeutic agent or procedure.

Therapeutic agents that may be administered in combination with a low molecular weight over-sulfated polysaccharide described herein, or a composition thereof, may be selected among a large variety of biologically active compounds that are known to have a beneficial effect in the management of a MPS disorder, in particular a MPS III disorder (e.g., anti-inflammatory agents, immunomodulatory agents, analgesics, antimicrobial agents, antibacterial agents, antibiotics, antioxidants, antiseptic agents, anti-seizure medications, medications for heart problems, and combinations thereof). Therapeutic procedures that may be performed in combination with administration of a low molecular weight over-sulfated polysaccharide, or a composition thereof, include, but are not limited to, orthopedic surgery for correcting joint abnormalities, corneal transplant for vision abnormalities, correction of hearing defects, and the like. Other therapies used to manage the symptoms of Sanfilippo syndrome include speech therapy, occupational therapy, physical therapy, behavioral therapy, and the like.

### 2—Administration

A low molecular weight over-sulfated polysaccharide described herein (optionally after formulation with one or more appropriate pharmaceutically acceptable carriers or excipients), in a desired dosage, can be administered to a subject in need thereof by any suitable route. Various delivery systems are known and can be used to administer an exopolysaccharide derivative of the present invention, including tablets, capsules, injectable solutions, encapsulation in liposomes, microparticles, microcapsules, etc. Methods of administration include, but are not limited to, dermal, intradermal, intramuscular, intraperitoneal, intralesional, intravenous, subcutaneous, intranasal, pulmonary, epidural, ocular, and oral routes. A low molecular weight over-sulfated polysaccharide described herein, or a composition thereof, may be administered by any convenient or other appropriate route, for example, by infusion or bolus injection, by adsorption through epithelial or mucocutaneous linings (e.g., oral, mucosa, rectal and intestinal mucosa, etc). Administration can be systemic or local. Parenteral administration may be directed to a given tissue of the patient, such as by catheterization. As will be appreciated by those of ordinary skill in the art, in embodiments where the low molecular weight over-sulfated polysaccharide is administered along with an additional therapeutic agent, the exopolysaccharide derivative and the therapeutic agent may be administered by the same route (e.g., orally) or by different routes (e.g., orally and intravenously).

### 3—Dosage

Administration of a low molecular weight over-sulfated polysaccharide as described herein (or a composition thereof) according to the present invention will be in a dosage such that the amount delivered is effective for the intended purpose. The route of administration, formulation and dosage administered will depend upon the therapeutic effect desired, the severity of the disorder being treated, the presence of any infection, the age, sex, weight and general health condition of the patient as well as upon the potency, bioavailability and in vivo half-life of the low molecular weight over-sulfated polysaccharide, the use (or not) of concomitant therapies, and other clinical factors. These factors are readily determinable by the attending physician in the course of the therapy. Alternatively, or additionally, the dosage to be administered can be determined from studies using animal models. Adjusting the dose to achieve maximal efficacy based on these or other methods is well known in the art and is within the capabilities of trained physicians. As studies are conducted using a low molecular weight over-sulfated polysaccharide described herein, further information will emerge regarding the appropriate dosage levels and duration of treatment.

A treatment according to the present invention may consist of a single dose or multiple doses. Thus, administration of a low molecular weight over-sulfated polysaccharide described herein, or a composition thereof, may be constant for a certain period of time or periodic and at specific intervals, e.g., hourly, daily, weekly (or at some other multiple day interval), monthly, yearly (e.g., in a time release form). Alternatively, the delivery may occur at multiple times during a given time period, e.g., two or more times per week, two or more times per month, and the like. The delivery may be continuous delivery for a period of time, e.g., intravenous delivery.

### III—Pharmaceutical Compositions

As mentioned above, a low molecular weight over-sulfated polysaccharide described herein may be administered per se or as a pharmaceutical composition. Accordingly, the present invention provides pharmaceutical compositions comprising an effective amount of a low molecular weight over-sulfated polysaccharide (in particular HES5.1 or GYS8) and at least one pharmaceutically acceptable carrier or excipient. In some embodiments, the composition further comprises one or more additional biologically active agents.

A low molecular weight over-sulfated polysaccharide described herein, or pharmaceutical compositions thereof, may be administered in any amount and using any route of administration effective for achieving the desired prophylactic or therapeutic effect. The optimal pharmaceutical formulation can be varied depending upon the route of administration and desired dosage. Such formulations may influence the physical state, stability, rate of in vivo release, and rate of in vivo clearance of the administered active ingredient.

The pharmaceutical compositions of the present invention may be formulated in dosage unit form for ease of administration and uniformity of dosage. The expression “unit dosage form”, as used herein, refers to a physically discrete unit of for the patient to be treated. It will be understood, however, that the total daily dosage of the compositions will be decided by the attending physician within the scope of sound medical judgement.

### 1—Formulation

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a non-toxic parenterally acceptable diluent or solvent, for example, as a solution in 2,3-butanediol. Among the acceptable vehicles and solvents that may be employed are water, Ringer's solution, U.S.P. and isotonic sodium chloride solution. In addition, sterile, fixed oils are conventionally employed as a solution or suspending medium. For this purpose, any bland fixed oil can be employed including synthetic mono- or di-glycerides. Fatty acids such as oleic acid may also be used in the preparation of injectable formulations. Sterile liquid carriers are useful in sterile liquid form compositions for parenteral administration.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Liquid pharmaceutical compositions which are sterile solutions or suspensions can be administered, for example, by intravenous, intramuscular, intraperitoneal or subcutaneous injection. Injection may be via single push or by gradual infusion. Where necessary or desired, the composition may include a local anaesthetic to ease pain at the site of injection.

In order to prolong the effect of an active ingredient, it is often desirable to slow the absorption of the ingredient from subcutaneous or intramuscular injection. Delaying absorption of a parenterally administered active ingredient may be accomplished by dissolving or suspending the ingredient in an oil vehicle. Injectable depot forms are made by forming micro-encapsulated matrices of the active ingredient in biodegradable polymers such as polylactide-polyglycolide. Depending upon the ratio of active ingredient to polymer and the nature of the particular polymer employed, the rate of ingredient release can be controlled. Examples of other biodegradable polymers include poly(orthoesters) and poly(anhydrides). Depot injectable formulations can also be prepared by entrapping the active ingredient in liposomes or microemulsions which are compatible with body tissues.

Liquid dosage forms for oral administration include, but are not limited to, pharmaceutically acceptable emulsions, microemulsions, solutions, suspensions, syrups, elixirs, and pressurized compositions. In addition to a low molecular weight over-sulfated polysaccharide described herein, the liquid dosage form may contain inert diluents commonly used in the art such as, for example, water or other solvent, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cotton seed, ground nut, corn, germ, olive, castor, and sesame oils), glycerol, tetrahydrofurfuryl alcohol, polyethylene glycols, and fatty acid esters of sorbitan and mixtures thereof. Besides inert diluents, the oral compositions can also include adjuvants such as wetting agents, suspending agents, preservatives, sweetening, flavouring, and perfuming agents, thickening agents, colors, viscosity regulators, stabilizes or osmo-regulators. Examples of suitable liquid carriers for oral administration include water (potentially containing additives as above, e.g., cellulose derivatives, such as sodium carboxymethyl cellulose solution), alcohols (including monohydric alcohols and polyhydric alcohols such as glycols) and their derivatives, and oils (e.g., fractionated coconut oil and arachis oil). For pressurized compositions, the liquid carrier can be halogenated hydrocarbon or other pharmaceutically acceptable propellant.

Solid dosage forms for oral administration include, for example, capsules, tablets, pills, powders, and granules. In such solid dosage forms, the low molecular weight over-sulfated polysaccharide described herein may be mixed with at least one inert, pharmaceutically acceptable excipient or carrier such as sodium citrate or dicalcium phosphate and one or more of: (a) fillers or extenders such as starches, lactose, sucrose, glucose, mannital, and silicic acid; (b) binders such as, for example, carboxymethylcellulose, alginates, gelatine, polyvinylpyrrolidone, sucrose, and acacia; (c) humectants such as glycerol; (d) disintegrating agents such as agar-agar, calcium carbonate, potato or tapioca starch, alginic acid, certain silicates, and sodium carbonate; (e) solution retarding agents such as paraffin; absorption accelerators such as quaternary ammonium compounds; (g) wetting agents such as, for example, cetyl alcohol and glycerol monostearate; (h) absorbents such as kaolin and bentonite clay; and (i) lubricants such as talc, calcium stearate, magnesium stearate, solid polyethylene glycols, sodium lauryl sulfate, and mixtures thereof. Other excipients suitable for solid formulations include surface modifying agents such as non-ionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidal silicon dioxide, phosphates, sodium dodecylsulfate, magnesium aluminium silicate, and triethanolamine. In the case of capsules, tablets and pills, the dosage form may also comprise buffering agents.

Solid compositions of a similar type may also be employed as fillers in soft and hard-filled gelatine capsules using such excipients as lactose or milk sugar as well as high molecular weight polyethylene glycols and the like. The solid dosage forms of tablets, capsules, pills, and granules can be prepared with coatings and shells such as enteric coatings, release controlling coatings and other coatings well known in the pharmaceutical formulating art. They may optionally contain opacifying agents and can also be of a composition such that they release the active ingredient(s) only, or preferably, in a certain part of the intestinal tract, optionally, in a delaying manner. Examples of embedding compositions which can be used include polymeric substances and waxes.

In certain embodiments, it may be desirable to administer an inventive composition locally to a specific area. This may be achieved, for example, and not by way of limitation, by local infusion, topical application, by injection, by means of a catheter, by means of suppository, or by means of a skin patch or stent or another implant.

For topical administration, the composition is preferably formulated as a gel, an ointment, a lotion, or a cream which can include carriers such as water, glycerol, alcohol, propylene glycol, fatty alcohols, triglycerides, fatty acid esters, or mineral oil. Other topical carriers include liquid petroleum, isopropyl palmitate, polyethylene glycol, ethanol (95%), polyoxyethylenemonolaurat (5%) in water, or sodium lauryl sulfate (5%) in water. Other materials such as antioxidants, humectants, viscosity stabilizers, and similar agents may be added as necessary.

In addition, in certain instances, it is expected that the inventive compositions may be disposed within transdermal devices placed upon, in, or under the skin. Such devices include patches, implants, and injections which release the active ingredient by either passive or active release mechanisms. Transdermal administrations include all administration across the surface of the body and the inner linings of bodily passage including epithelial and mucosal tissues. Such administrations may be carried out using the present compositions in lotions, creams, foams, patches, suspensions, solutions, and suppositories.

Transdermal administration may be accomplished through the use of a transdermal patch containing an active ingredient (i.e., a low molecular weight over-sulfated polysaccharide described herein) and a carrier that is non-toxic to the skin and allows the delivery of the ingredient for systemic absorption into the bloodstream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may be suitable. A variety of occlusive devices may be used to release the active ingredient into the bloodstream such as a semi-permeable membrane covering a reservoir containing the active ingredient with or without a carrier, or a matrix containing the active ingredient.

Suppository formulations may be made from traditional materials, including cocoa butter, with or without the addition of waxes to alter the suppository's melting point, and glycerine. Water soluble suppository bases, such as polyethylene glycols of various molecular weights, may also be used.

Materials and methods for producing various formulations are known in the art and may be adapted for practicing the subject invention. Suitable formulations for the delivery of antibodies can be found, for example, in “Remington's Pharmaceutical Sciences”, E. W. Martin, 18^{th} Ed., 1990, Mack Publishing Co.: Easton, Pa.

### 2—Additional Biological Active Agents

In certain embodiments, the low molecular weight over-sulfated polysaccharide described herein is the only active ingredient in a pharmaceutical composition of the present invention. In other embodiments, the pharmaceutical composition further comprises one or more biologically active agents. Examples of suitable biologically active agents include, but are not limited to, anti-inflammatory agents, immunomodulatory agents, analgesics, antimicrobial agents, antibacterial agents, antibiotics, antioxidants, antiseptic agents, anti-seizure medications, medications for heart problems, and combinations thereof.

In such pharmaceutical compositions, the low molecular weight over-sulfated polysaccharide described herein and the at least one additional therapeutic agent may be combined in one or more preparations for simultaneous, separate or sequential administration of the low molecular weight over-sulfated polysaccharide and therapeutic agent(s). More specifically, an inventive composition may be formulated in such a way that the low molecular weight over-sulfated polysaccharide and therapeutic agent(s) can be administered together or independently from each other. For example, the low molecular weight over-sulfated polysaccharide and a therapeutic agent can be formulated together in a single composition. Alternatively, they may be maintained (e.g., in different compositions and/or containers) and administered separately.

### 3—Pharmaceutical Packs or Kits

In another aspect, the present invention provides a pharmaceutical pack or kit comprising one or more containers (e.g., vials, ampoules, test tubes, flasks or bottles) containing one or more ingredients of an inventive pharmaceutical composition, allowing administration of a low molecular weight over-sulfated polysaccharide described herein.

Different ingredients of a pharmaceutical pack or kit may be supplied in a solid (e.g., lyophilized) or liquid form. Each ingredient will generally be suitable as aliquoted in its respective container or provided in a concentrated form. Packs or kits according to the invention may include media for the reconstitution of lyophilized ingredients. Individual containers of the kits will preferably be maintained in close confinement for commercial sale.

In certain embodiments, a pack or kit includes one or more additional therapeutic agent(s). Optionally associated with the container(s) can be a notice or package insert in the form prescribed by a governmental agency regulating the manufacture, use or sale of pharmaceutical or biological products, which notice reflects approval by the agency of manufacture, use or sale for human administration. The notice of package insert may contain instructions for use of a pharmaceutical composition according to methods of treatment disclosed herein.

An identifier, e.g., a bar code, radio frequency, ID tags, etc., may be present in or on the kit. The identifier can be used, for example, to uniquely identify the kit for purposes of quality control, inventory control, tracking movement between workstations, etc.

Further aspects and advantages of this invention will be disclosed in the following figures and examples, which should be regarded as illustrative and not limiting the scope of this application.

### EXAMPLES

The following examples describe some of the preferred modes of making and practicing the present invention. However, it should be understood that the examples are for illustrative purposes only and are not meant to limit the scope of the invention. Furthermore, unless the description in an Example is presented in the past tense, the text, like the rest of the specification, is not intended to suggest that experiments were actually performed, or data were actually obtained.

### Working Hypothesis

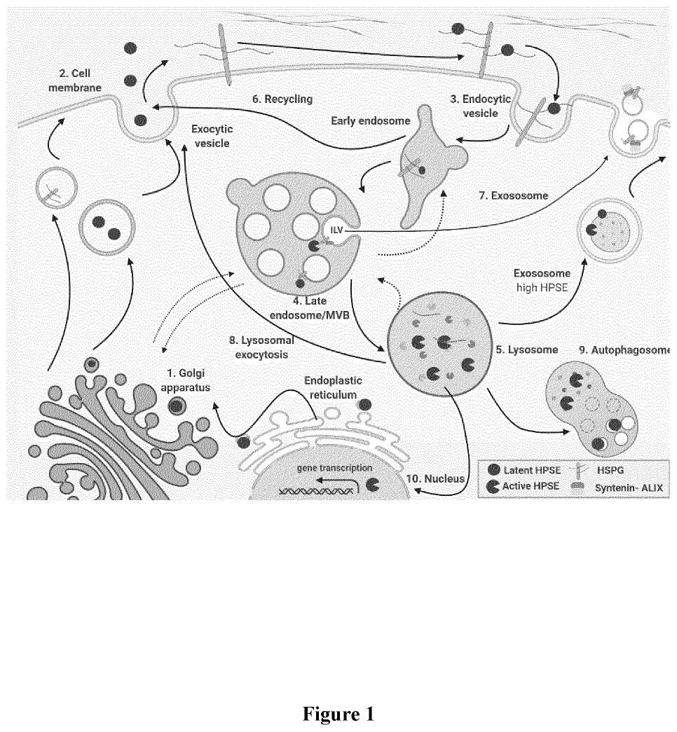

FIG. 1 presents a schematic model of herapan sulfate (HS) proteoglycans and heparanase (HPSE) trafficking. In this scheme: 1. In the Golgi apparatus, HS chains are polymerized and pre-HPSE is processed to produce pro-HPSE by the elimination of the N-terminal signal peptide. 2. The newly biosynthesized HSPGs are then shifted to the cell membrane where they can interact with the pro-HPSE and 3. the complex is rapidly internalized by endocytosis and then 4. accumulated in the late endosome. 5. Upon fusion of the late endosome with lysosome, pro-HPSE is activated and cleaves HS chains that are completely degraded by lysosomal hydrolases. 6. HPSE and HSPGs can be recycled to the cell surface from endosomes. It appears that active HPSE pursues other paths in the cells. 7. Trimming of HS from syndecans by active HPSE present in late endosomes leads to formation of the syndecan-syntenin-ALIX complex. Intraluminal vesicles (ILV) are then formed by the invagination of endosomal membranes, resulting in the formation of multivesicular bodies (MVBs). MVBs release ILVs as exosomes upon fusion with the cell membrane and deliver their cargo to recipient cells. In the presence of high levels of HPSE, the enzyme can be found on the surface of exosomes and modulates tumor microenvironment. 8. Lysosomal exocytosis has been observed in malignant cells. 9. HPSE also regulates autophagy by driving fusion of lysosomes with autophagosomes which degrade macromolecules into monomeric units. 10. Perinuclear lysosomal HPSE can also translocate into the nucleus and regulate gene transcription and cell differentiation.

The working hypothesis was that inhibition of HPSE by exopolysaccharide derivatives can modify the catabolic fate of HS in Sanfilippo syndrome, by preventing lysosomal degradation of HPSE-cleaved fragments of HS in a context of defective degradation enzymes.

### Exopolysaccharide Derivatives

The bacterial GY785 and HE800 exopolysaccharides (EPSs) were produced, purified and characterized as previously described (Guezennec et al., Carbohydr. Polym., 1998, 37: 19-24). The preparation, purification and characterization of low molecular weight over-sulfated EPS derivatives were performed as previously reported (Ruiz Velasco et al., Glycobiology, 2011, 21: 781-795; WO 2006/003290; Colliec-Jouault et al., Biochim. Biophys. Acta, 2001, 1528: 141-151; WO 2007/066009; WO 02/02051; Guezennec J. et al. in Carbohydrate Polymers 1998, 37(1): 19-24; Senni et al., Mar. Drugs, 2013, 11: 1351-1369; Merceron et al., Stem Cells, 2012, 30: 471-480; Senni et al., Mar. Drugs, 2011, 9: 1664-1681; Heymann et al., Molecules, 2016, 21: 309). Briefly, native high molecular weight GY785 EPS and HEP800 were depolymerized first using a free-radical depolymerisation process to obtain low molecular weight derivatives with different molecular weights. These low molecular weight GY785 and HE800 EPS derivatives were then sulfated in dimethylformamide (DMF) using pyridine sulfate as sulfating agent leading to low molecular weight over-sulfated polysaccharides. Molecular weights (MWs) before and after sulfation were determined by HPSEC-MALS and sulfur content (wt % S) by HPAEC chromatography. ATR-FTIR and NMR spectroscopy were used to assess the efficiency of sulfation reaction. Heparin sodium salt from porcine intestinal mucosa H4784 was purchased from Sigma.

### Example 1: Anti-Coagulant and Anti-Heparanase Activities

The endoglucuronidase heparanase (HPSE) is the first enzyme to degrade heparan sulfate (HS) in its catabolic pathway, cleaving large segments of the HS chain that are subsequently depolymerized in lysosomes by exoglycosidases. HS-6-O-sulfatase 1 and 2 (Sulf1 and Sulf2) remove sulfate groups from glucosamine units, which modifies the charge distribution in HS, and impacts their interactions with ligand proteins, such as growth factors. The goal of the first experiment was to determine whether the low molecular weight over-sulfated EPS derivatives were able to inhibit the enzymes modifying HS chains post-synthetically, heparanase and sulfatase.

### Results

Anti-Coagulant and Anti-Heparanase Activities of Exopolysaccharide Derivatives. In collaboration with Dr. Jin-ping Li (Uppsala University, Sweden), the Inventors have determined the anti-heparanase (anti-HPSE) activity of different exopolysaccharide (EPS) derivatives. The results are presented in Table 1 below. The anti-HPSE activity was found to be dependent on the charge of the EPS derivative but was not significantly affected by the size of the EPS derivatives comprised between 5,000 to 16,000 Da. IC₅₀ values for the EPS derivatives were determined to be between 1 and 5 μM (see Table 1). Consequently, the EPS derivatives tested exhibit a strong potential as HPSE inhibitors.

In addition, in collaboration with Dr. Romain Vivès (Structure and Activités des Glycosaminoglycanes, Institut de Biologie Structurale de Grenoble), the present Inventors have found that the EPS derivatives studies also inhibit the 6 endosulfatases (Sulf1 and Sulf2), enzymes that modify the sulfation pattern of HS and thus modulate their affinity for ligand proteins.

TABLE 1
Anti-coagulant and anti-HPSE activities of the EPS derivatives.
The best candidates for MPS treatment are in bold.

| | | | | Anti-HPSE | Anti-HPSE | μg/ml for | μg/ml for |
|---|---|---|---|---|---|---|---|
| EPS | | MW | % | IC₅₀ | IC₅₀ | doubling | doubling |
| deriv. | Name | (g/mol) | sulfate | (μM) | (μg/ml) | aPTT | aPTT |
| HES34 | A5 | 34,000 | 0 | — | — | 80 | 100 |
| HES16 | A6 | 16,000 | 30 | 3.45 | 55.2 | 2 | 4 |
| | | | | (1.1-5.8) | (17.6-92.8) | | |
| HES5.1 | A5_3 | 5,100 | 50 | 3.55 | 18.1 | 10 | 50 |
| | | | | (1.8-5.3) | (9.2-27.0) | | |
| HES3.7 | A5_1 | 3,700 | 0 | — | — | >200 | >200 |
| GYS21 | A7 | 21,000 | 10 | — | — | >200 | >200 |
| GYS15 | 8A | 15,000 | 45 | 0.09 | 1.35 | 20 | 10 |
| GYS8 | A5_4 | 8,000 | 36 | 2.9 | 23.2 | 10 | 100 |
| | | | | (2.1-3.7) | (16.8-29.6) | | |
| GYS4 | A5_2 | 4,000 | 10 | — | — | >200 | >200 |

### Example 2: Marine Bacterial Exopolysaccharide Derivatives as Potential Treatment for Sanfilippo Syndrome

HES5.1 (A5_3) and GYS8 (A5_4), the two low molecular weight over-sulfated polysaccharides found to exhibit the most interesting properties in Example 1 were further investigated to explore their ability to serve as treatment for Sanfilippo treatment. Based to the working hypothesis according to which inhibition of the first cleavage of HS chains prevents lysosomal degradation of the fragments, the exopolysaccharide derivatives should be active to treat all forms of Sanfilippo syndrome (MPSIII). The Inventors studied one of the MPSIII disorders, MPS III subtype A (MPSIIIA), where the enzyme HS-sulfamidase is inactivated by mutations. According to the hypothesis, inhibition of HPSE by the low molecular weight over-sulfated polysaccharide should modify the catabolic fate of HS, by preventing lysosomal degradation of HPSE-cleaved fragments in a context of defective degradation enzymes.

The present Inventors worked in vitro using fibroblasts derived from a mouse model of MPSIIIA (in collaboration with Dr. K. Hemsley, Adelaide, Australia) (Crawley et al., Brain Res., 2006, 1104: 1-17). They purified HS in the extracellular compartment (medium plus surface) and in the intracellular compartment and characterized their size and charge by gel electrophoresis (PAGE) and by gel filtration (GFC). They analyzed the impact of treatment with a low molecular weight over-sulfated polysaccharide on these parameters. For each of the exopolysaccharide derivatives, two independent experiments were performed, starting from the seeding of cells to the isolation of HS and final analysis of the dimensions (length/molecular weight) of HS.

### Results

Analysis of the Amount of Heparan Sulfate (HS). In each experiment, cells were counted, and the results obtained were normalized to the total number of cells. In order to further normalize results, correction for the ³⁵SO₄ incorporation level was performed, and was calculated as the ratio between recovered measured total radioactivity and the initial radioactivity administered to cells. The incorporation level in the present experiments was found to span from 1.2% to 5.6%.

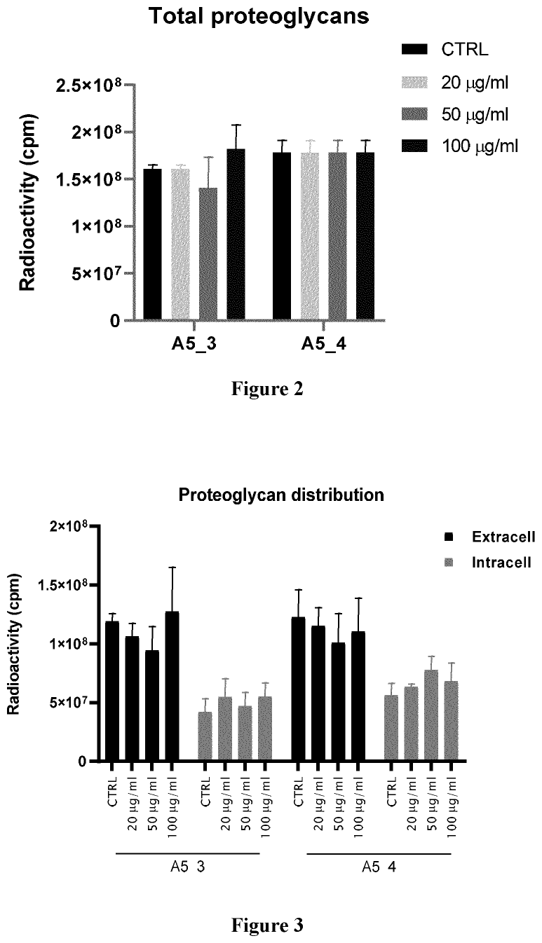

Observations of the Effects on Proteoglycans: The first purification step, consisting in a size exclusion chromatography, allowed to isolate high molecular weight species from free Na₂³⁵SO₄. The total amount of sulfated proteoglycans (PGs) is considered as the recovered material from the first purification step and is expressed in cpm (radioactivity). For both exopolysaccharide derivatives (HES5.1 (A5_3) and GYS8 (A5_4)), the total amount of PGs was found to be similar in control untreated MPSIIIA cells and in treated MPSIIIA cells (see FIG. 2).

By taking a deeper look into the distribution of PGs, the Inventors observed a higher amount in proteoglycans in the extracellular compartment than in the corresponding intracellular one (see FIG. 3). Variations between control and treated cells are not significant, meaning the EPS derivative treatment does not impact the total amount and distribution of PGs produced by the cells.

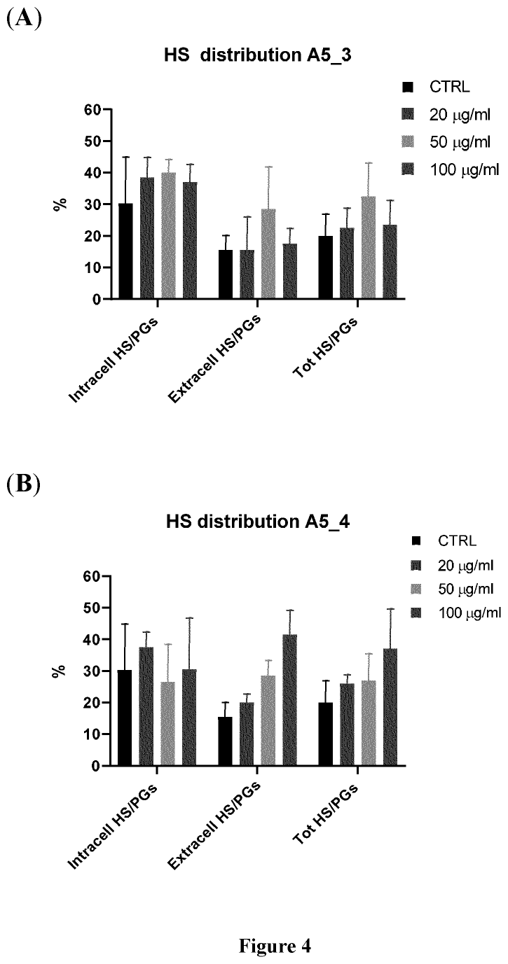

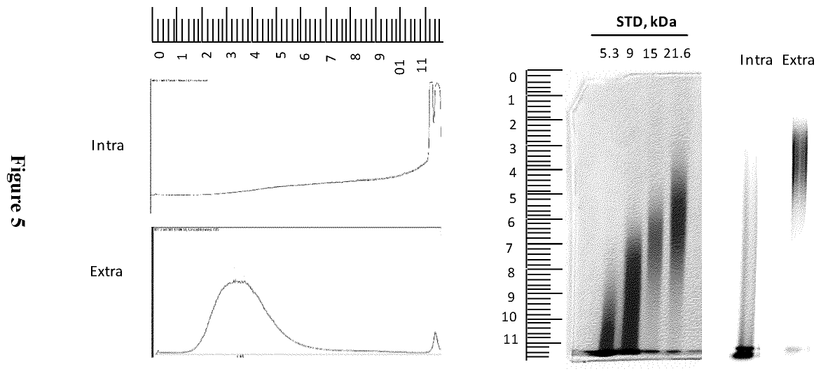

Observations of the Effects on Heparan Sulfate: Heparan sulfate was isolated from both extracellular and intracellular fractions and it represented a minority of total glycosaminoglycans present in the cells. In fact, the percentage of HS in total PGs was found to span from 12% to 37% in control cells. The Inventors calculated the percentage of HS with respect to the PGs in the corresponding fraction, considering 100% as the amount of PGs (see FIG. 4). In control cells, HS was determined to represent (29±11)% of intracellular PGs, and (16±4)% of extracellular PGs and (20±5)% of total PGs (n=8). In general, both the repartition of HS and the total percentage of HS vary but there is no statistical difference between control and treated cells. There is a trend toward the increase in HS in the extracellular compartment when cells are treated with A5_4 (GYS8) but not with A5_3 (HES5.1).

In summary, it can be concluded that treatment of MPSIIIA cells with the exopolysaccharide derivatives does not alter the total amount and distribution of proteoglycans. The percentage of total heparan sulfate although subjected to variation is not significantly affected by EPS derivative treatment.

The following step in the analysis was to look at the dimensions (length/molecular weight) of heparan sulfate, with the idea that treatment with a low molecular weight over-sulfated polysaccharide would impact the first step of HS degradation and therefore, HS chains with higher molecular weight would be found in the intracellular compartment.

Analysis of the Molecular Weight of Heparan Sulfate (HS) in Control Cells. Wild type cells produce full size HS, with a molecular weight above 20 kDa, which is assembled in the intracellular compartment and expressed on the cell surface as proteoglycans (Colliec-Jouault et al., J. Biol. Chem., 1994, 269: 24953-24958). The lysosomal catabolism of HS is fast and low molecular weight (LMW) intermediates are transient and in low amounts that are not detected (Yanagishita and Hascall, Proteoglycan metabolism by rat ovarian granulosa cells in vitro. In: Wight, Mecham RP (eds.), Biology of the proteoglycans, 1 ed. Orlando: Academic Press; 1987: 105-128). In the MPSIIIA cells, the intracellular HS exhibited a widely spread out distribution of LMW, partially degraded, HS fragments that accumulate in lysosomes due to the dysfunctional sulfamidase, as confirmed by PAGE-NaCl electrophoresis (see FIG. 5). By comparison of samples with glycosaminoglycan standards (Mulloy et al., Thromb Haemost. 1997, 77(4): 668-674), it was possible to estimate the molecular weight of extracellular HS which is concentrated in the high molecular weight region (>20 kDa). In contrast, a wide range of HS fragments that accumulate in the LMW region (<20 kDa) can be observed for intracellular HS, resulting in a smear on the gel.

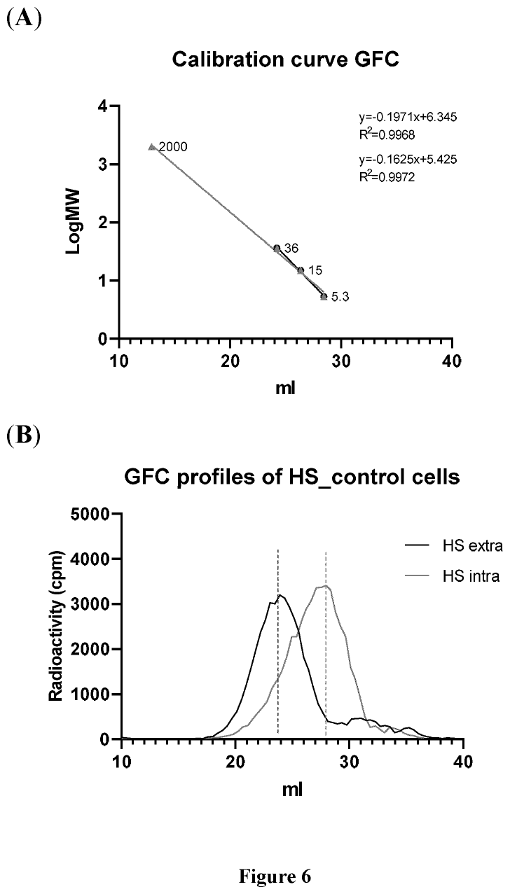

The Inventors then decided to use a different technique and opted for gel filtration chromatography (GFC) on a CL6B resin equilibrated in PBS, 0.15M NaCl, pH 7.4. Separation of molecules by PAGE depends not only on the molecular weight but also on the overall negative charge, while separation by GFC depends mostly on the dimension of the molecules. FIG. 6 shows the calibration curves obtained with the standards and an example of the profiles of control intracellular and extracellular HS. A clear peak of intracellular HS was observed with GFC. The molecular weight and distribution are presented in Table 2. The average MW of extracellular HS calculated by PAGE (n=8) was found to be 37.1 kDa spanning from 24 to 50.7 kDa, while that calculated by GFC (n=3) was found to be 43 kDa spanning from 17.7 to 116.7 kDa. Intracellular HS was well resolved only with GFC, with an average MW of 7.9 kDa but fragments spanned from 3.5 to 28 kDa (n=5).

TABLE 2
Size distribution of Extracellular and Intracellular HS in MPSIIIA
Cells. HS was isolated from control cells and analyzed by PAGE
and GFC. The molecular weight (MW) is expressed in kDa ± SD.

| | PAGE-NaCl | | GFC | |
|---|---|---|---|---|
| | MW peak | MW range | MW peak | MW range |
| Extracellular | 37.1 ± 2.3 | 24-50.7 | 43 ± 8.6 | 17.7-116.7 |
| Intracellular | smear | — | 7.9 ± 1.8 | 3.5-28 |

The range was calculated at half peak height.

Analysis of the Molecular Weight of Heparan Sulfate (HS) in Treated Cells. The effect of a treatment with a low molecular weight over-sulfated polysaccharide on MPSIIIA cells was to block the partial degradation of intracellular HS so that HS from treated cells remains undegraded and the MW is similar to the extracellular species (>20 kDa). Three concentrations of EPS derivative were tested: 20, 50 and 100 μg/ml. Cells were treated for four days before adding the radioactive sulfate donor and were collected after 24 hours.

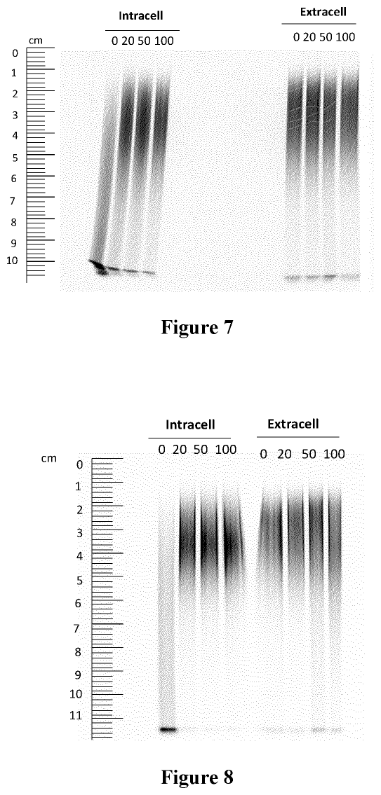

FIG. 7 and FIG. 8 show examples of PAGE NaCl gels obtained in control and A5_3 (HES5.1)-treated cells and in control and A5_4 (GYS8)-treated cells, respectively. Control cells show intracellular low molecular weight HS and extracellular high molecular weight HS. Treated cells clearly exhibit high molecular weight intracellular HS (>20 kDa) compared to untreated cells. The molecular weight of treated and untreated extracellular HS is the same. The observed effect was similar for the three concentrations used.

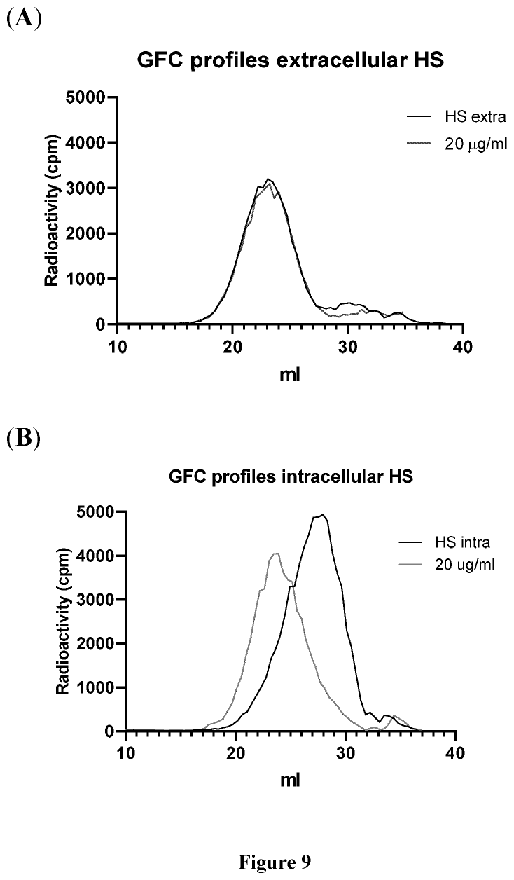

FIG. 9 shows GFC profiles of HS from control and treated (20 μg/ml of A5_3 (HES5.1)) cells. Extracellular HS was not affected by treatment, as observed by the superimposed GFC profiles. The treatment affects intracellular HS: a shift towards higher MW (>20 kDa) was clearly observed upon treatment and the MW of treated HS was similar to extracellular high molecular weight HS.

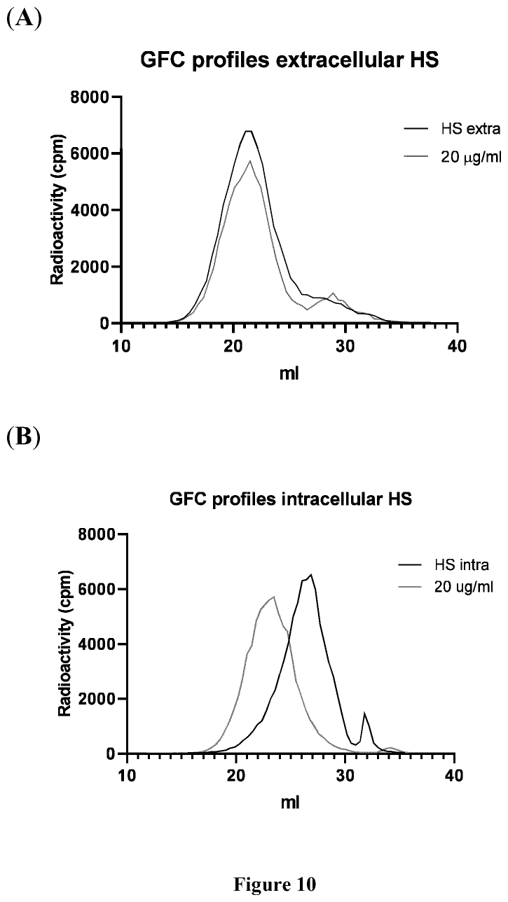

FIG. 10 shows GFC profiles of HS from control and treated (20 μg/ml of A5_4 (GYS8)) cells. The effect of A5_4 treatment is similar to A5_3: extracellular HS is not affected by treatment with a low molecular weight over-sulfated polysaccharide, while intracellular HS shifts towards higher MW (>20 kDa).

Tables 3 and 4 summarize the results of both PAGE and GFC analyses. Both techniques demonstrate the specificity of treatment that affects only intracellular HS. The effect is similar for all three concentrations tested.

TABLE 3
Size distribution of HS in MPSIIIA cells treated by
A5_3 (HES5.1). HS was isolated from control and treated
cells and analyzed by PAGE and GF. The MW is expressed in kDa.

| | | * PAGE-NaCl | | GFC | |
|---|---|---|---|---|---|
| | Sample | MW peak | MW range | MW peak | MW range |
| Intra- | untreated | smear | smear | 9.2 | 5.5-39.6 |
| cellular | 20 μg/ml | 33.5 ± 1.1 | 18.8-51.1 | 36.5 ± 2.7 | 12.8-91.8 |
| | 50 μg/ml | 34.8 ± 1.7 | 21.1-54.1 | | |
| | 100 μg/ml | 36.2 ± 2.7 | 22.5-55.2 | | |
| Extra- | untreated | 43.1 ± 12.1 | 32.1-56.8 | 52.7 | 20.5-135.2 |
| cellular | 20 μg/ml | 42.2 | 24.2-60.3 | 49.6 | 20.2-141.5 |
| | 50 μg/ml | 43.3 ± 11.3 | 28.4-61.0 | | |
| | 100 μg/ml | 46.2 ± 12.2 | 30-62.2 | | |

* Average of two independent experiments ± SD.
The range was calculated at half peak height.

TABLE 4
Size distribution of HS in MPSIIIA cells treated by
A5_4 (GYS8). HS was isolated from control and treated
cells and analyzed by PAGE and GF. The MW is expressed in kDa.

| | | * PAGE-NaCl | | GFC | |
|---|---|---|---|---|---|
| | Sample | MW peak | MW range | MW peak | MW range |
| Intra- | untreated | smear | smear | 7.75 ± 3.2 | 2.9-23.45 |
| cellular | 20 μg/ml | 34.9 ± 5 | 23.4-50.8 | 40.6 ± 0.1 | 16.2-113 |
| | 50 μg/ml | 34.2 ± 6.2 | 23.7-50.6 | | |
| | 100 μg/ml | 34.6 ± 6.3 | 23.9-50.5 | | |
| Extra- | untreated | 35.6 ± 2.4 | 24.2-47.9 | 52.7 | 20.5-135.2 |
| cellular | 20 μg/ml | 35.5 ± 0.7 | 24.9-49.2 | 49.6 | 20.2-141.5 |
| | 50 μg/ml | 41.1 ± 4.3 | 23.7-50.2 | | |
| | 100 μg/ml | 32.1 ± 2.1 | 21.3-49.9 | | |

* Average of two independent experiments ± SD.
The range was calculated at half peak height.

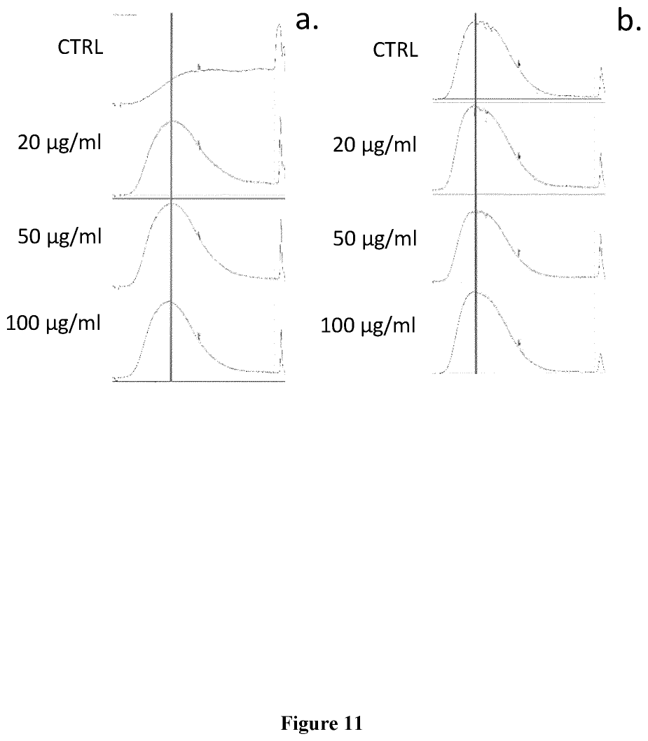

Another way of expressing the MW distribution of HS is to look at the percentage of chains before and after a certain threshold. For simplicity, the inventors defined it as the peak of extracellular HS in PAGE profiles, which corresponds to the peak of intracellular HS upon treatment, as indicated in FIG. 11 and Table 5. Since the effect was found to be similar for the three concentrations tested, the results were expressed as the mean of the three concentrations. Clearly, the percentage of high molecular weight HS after treatment with either A5_3 (HES5.1) or A5_4 (GYS8) reaches the value of extracellular control HS.

TABLE 5
Percentage MW distribution of HS in MPSIIIA cells treated with
EPS derivatives. HS was isolated from control and treated
cells and analyzed by PAGE. It is reported the % area below
the threshold. Control is untreated cells (n = 8), while
treated is the mean of the three concentrations used (n = 6).

% area < threshold

| | intracellular | extracellular |
|---|---|---|
| Control | 11 ± 4 | 37 ± 7 |
| A5_3 (HES5.1) | 39 ± 5 | 42 ± 1 |
| A5_4 (GYS8) | 37 ± 6 | 37 ± 6 |

### CONCLUSIONS

Both A5_3 (HES5.1) and A5_4 (GYS8) proved to be able to affect HS turnover thus leading to an incomplete degradation of intracellular HS. A similar effect was observed when cells were treated with different concentrations of each of the low molecular weight over-sulfated polysaccharide, indicating that not only 20 μg/ml of EPS derivative are effective but probably the concentration can be further lowered. According to the initial working hypothesis, intact HS chains, that are not cleaved by HPSE, cannot enter the lysosomal degradative pathway and are redirected to the extracellular space with clearance in blood and urine. Thus, cells can be preserved from toxic lysosomal HS accumulation. Consequently, both A5_3 (HES5.1) and A5_4 (GYS8) have a strong potential for the treatment of Sanfilippo syndrome patients. Treatment with these EPS derivatives could alleviate the symptoms due to lysosomal overload with incompletely degraded heparan sulfate.

Throughout this application, various references describe the state of the art to which this invention pertains. The disclosures of these references are hereby incorporated by reference into the present disclosure.

1-11. (canceled)

12. A method for preventing or treating a mucopolysaccharidosis in a subject, the method comprising a step of administering, to the subject in need thereof, an effective amount of a low molecular weight over-sulfated polysaccharide with anti-heparanase activity, or a pharmaceutical composition thereof, wherein said low molecular weight over-sulfated polysaccharide is a derivative of a native exopolysaccharide (EPS) excreted by a mesophilic marine bacterium from a deep-sea hydrothermal environment and wherein said low molecular weight over-sulfated polysaccharide with anti-heparanase activity is obtained using a method comprising the following steps:
(a) a step consisting of free-radical depolymerization of the marine native EPS from the strain GY785 of the Alteromonas genus or from the strain HE800 of the Vibrio diabolicus genus so as to obtain a depolymerized EPS having a molecular weight of 5,000 to 100,000 g/mol;
(b) a subsequent step consisting of sulfation of the depolymerized EPS to obtain an over-sulfated depolymerized EPS, comprising adding to the depolymerized EPS at least one sulfation agent in an amount sufficient to obtain a sulfated polysaccharide having a degree of sulfate-group substitution of between 10% and 55% by weight relative to the total weight of the over-sulfated depolymerized EPS; and
(c) a subsequent step consisting of isolating the low molecular weight over-sulfated polysaccharide from the over-sulfated depolymerized EPS, wherein the low-molecular-weight over-sulfated polysaccharide has a molecular weight of between about 5,000 and about 16,000 g/mol.

13. The method according to claim 12, wherein in step (a) the free-radical depolymerization is performed on the native GY785 EPS excreted by the strain GY785 and wherein said low molecular weight over-sulfated polysaccharide has a molecular weight of between about 6 kDa and about 10 kDa or between about 7 kDa and about 9 kDa, and a degree of sulfate-group substitution of between about 30% and about 40% by weight relative to the total weight of the over-sulfated polysaccharide.

14. The method according to claim 13, wherein said low molecular weight over-sulfated polysaccharide is GYS8, which has a molecular weight of about 8 kDa, and a degree of sulfate-group substitution of about 36% by weight relative to the total weight of the over-sulfated polysaccharide.

15. The method according to claim 12, wherein in step (a) the free-radical depolymerization is performed on the native HE800 EPS excreted by the strain HE800 and wherein said low molecular weight over-sulfated polysaccharide has a molecular weight of between about 3 kDa and about 7 kDa or between about 4 kDa and about 6 kDa, and a degree of sulfate-group substitution of between about 45% and about 55% by weight relative to the total weight of the over-sulfated polysaccharide.

16. The method according to claim 15, wherein said low molecular weight over-sulfated polysaccharide is HE5.1, which has a molecular weight of about 5.1 kDa, and a degree of sulfate-group substitution of about 50% by weight relative to the total weight of the over-sulfated polysaccharide.

17. The method according to claim 12, wherein the step of isolating said low molecular weight over-sulfated polysaccharide from the over-sulfated depolymerized EPS is carried out by fractionation, in particular fractionation performed by size exclusion chromatography.

18. The method according to claim 12, wherein the mucopolysaccharidosis is a mucopolysaccharidosis type III.

19. The method according to claim 18, wherein the mucopolysaccharide type III is of subtype A, subtype B, subtype C or subtype D.

20. The method according to claim 12, wherein the pharmaceutical composition comprises a therapeutically effective amount of the low molecular weight over-sulfated polysaccharide with anti-heparanase activity and at least one pharmaceutically acceptable carrier or excipient.

21. The method according to claim 20, wherein said pharmaceutical composition further comprises one or more additional biologically active agents.

22. The method according to claim 21, wherein the one or more additional biologically active agents are selected from the group consisting of anti-inflammatory agents, immunomodulatory agents, analgesics, antimicrobial agents, antibacterial agents, antibiotics, antioxidants, antiseptic agents, anti-seizure medications, medications for heart problems, and combinations thereof.
